(19) United States
(12) Patent Application Publication
(10) Pub. No.: US 2020/0308633 A1
(43) Pub. Date: Oct. 1, 2020

(54) METHOD FOR TESTING PRESENCE OR ABSENCE OF MYCOPLASMA

(71) Applicant: TERUMO KABUSHIKI KAISHA, Tokyo (JP)

(72) Inventors: Risa KUSANAGI, Kanagawa (JP); Souta KIMURA, Kanagawa (JP); Kenichi SATOU, Kanagawa (JP)

(73) Assignee: TERUMO KABUSHIKI KAISHA, Tokyo (JP)

(21) Appl. No.: 16/795,780

(22) Filed: Feb. 20, 2020

Related U.S. Application Data
(63) Continuation of application No. PCT/JP2018/045034, filed on Dec. 7, 2018.

(30) Foreign Application Priority Data
Dec. 8, 2017 (JP) 2017-236416

(51) Int. Cl. C12Q 1/689; C12Q 1/04; C12N 15/10; C12N 1/20

(52) U.S. Cl. CPC C12Q 1/689; C12Q 1/04; C12N 2500/34; C12N 1/20; C12N 2500/32; C12N 15/1017

(57) ABSTRACT

A method for testing presence or absence of a Mycoplasma in a specimen is disclosed. The method includes: culturing a specimen; extracting a nucleic acid from the cultured specimen and performing amplification of the nucleic acid using a primer capable of amplifying a nucleic acid sequence derived from a Mycoplasma, in which the specimen is a biotechnology-related product or a part thereof.

### CROSS-REFERENCES TO RELATED APPLICATIONS

This application is a continuation of International Application No. PCT/JP2018/045034 filed on Dec. 7, 2018, and claims priority to Japanese Application No. 2017-236416 filed on Dec. 8, 2017, the entire content of both of which is incorporated herein by reference.

### REFERENCE TO SEQUENCE LISTING SUBMITTED AS A COMPLIANT ASCII TEXT FILE (.TXT)

Pursuant to the EFS-Web legal framework and 37 C.F.R. §§ 1.821-825 (see MPEP § 2442.03(a)), a Sequence Listing in the form of an ASCII-compliant text file (entitled “Sequence_Listing_1027550-001998_5T25,” created on 6 May, 2020, and 659 bytes in size) is submitted with the instant application, and the entire contents of the Sequence Listing are incorporated herein by reference.

### FIELD OF THE INVENTION

The present invention relates generally to a method for testing presence or absence of a Mycoplasma.

### BACKGROUND DISCUSSION

Mycoplasmas are well known as microorganisms which typically contaminate cell cultures. Mycoplasmas are microorganisms belonging to the class Mollicutes, are generally incapable of self-renewal without being parasitic in cells, and show polymorphism and filterability. In addition, Mycoplasmas have no cell wall and thus are resistant to β-lactam antibiotics such as penicillin. Detection of Mycoplasmas is generally difficult with typical microscope observation and contamination by Mycoplasmas is difficult to determine by visual inspection Thus, Mycoplasmas are cultured or subcultured without detection in many cases. Metabolic abnormality of cultured cells which are infected, cytokine induction, chromosomal variation, and the like are cited as factors influencing Mycoplasma contamination. In human bodies, it is known that a cytosolic membrane surface protein of a Mycoplasma acts as an antigen to affect the immune system by, for example, inducing inflammatory cytokines. Further, there is a concern that some Mycoplasmas cause diseases such as pneumonia, urethritis, and arthritis. Therefore, it is necessary to perform a Mycoplasma detection test in the fields of regenerative medicine using live cells or tissues, cell therapy not intended to regenerate organs or tissues (for example, cancer cell immunotherapy), cell or tissue processing products used for processing such as cultivation, activation, and differentiation induction, cell processed products, vaccines, antibody drugs, and the like, in which production is performed using biomedical materials derived from animals.

In The Japanese Pharmacopoeia 17th Edition (JP17), as the Mycoplasma detection test, a culturing method, a DNA staining method, and a nucleic acid amplification test (NAT) method are described. The culturing method needs a long culture period of 28 days in total, involving cultivation in a liquid culture medium for 14 days and subsequent cultivation in an agar medium for 14 days. The DNA staining method needs about seven days as a period of time necessary for a test, but has low sensitivity and needs 1 mL or more of a specimen. Further, in the nucleic acid amplification test method, a period of time necessary for a test is about several days, but typically needs about 1 to 10 mL of a specimen. Additionally, a method of replacing cultivation in an agar medium for 14 days in the culturing method with detection by polymerase chain reaction (PCR) has been reported (see “Detection of live Mycoplasmas by a combined method of preculture and PCR”, Annual Report of the National Veterinary Assay Laboratory, vol. 44, 13 to 20 (2007)). However, even in this method, cultivation for about 14 days is necessary and about 1 mL of a specimen is necessary.

### SUMMARY

In production of regenerative medicine products, there is a case where the amount of final products that can be produced may be limited, for example, the case of using autologous cells of patients, or the like. Further, the lifetime of the products is significantly shorter than general pharmaceutical products. Therefore, in regenerative medicine products, unlike general pharmaceutical products, it is desirable that the amount of a specimen necessary for a quality test is small and the period of time necessary for the test is also short. However, in a typical Mycoplasma detection test, as described above, the period of time necessary for the test in the culturing method is 28 days, that is, a longer period of time than the lifetime of products is required, a large amount, that is, 1 mL or more, of a specimen is required in the DNA staining method, the nucleic acid amplification test method, and an improved culturing method, and thus all of these methods are not preferable methods in the regenerative medicine field.

The present inventors have discovered as a part of researching a method for testing for the presence or absence of a Mycoplasma that by incorporating culturing a Mycoplasma before amplifying a nucleic acid in the nucleic acid amplification test method, existence of the Mycoplasma can be detected from a small amount of a specimen, thereby completing a new testing method of a Mycoplasma which can be advantageously used in regenerative medicine products. As a result of further research on such a method, the present inventors have encountered a new problem in that mixing of Mycoplasma nucleic acid fragments from a biological material or the like contained in products or testing materials which is not problematic in the culturing method and the DNA staining method may cause false positives. With respect to such problems, the present inventors have incorporated removing impurities before amplifying a nucleic acid and thus obtained new finding that false positives can be reduced. Research may then be continued based on such finding.

That is, a method disclosed here relates to the following.

[1] A method for testing presence or absence of a Mycoplasma in a specimen includes:
(a) a step of culturing a specimen; and
(b) a step of extracting a nucleic acid from the cultured specimen and performing amplification of the nucleic acid using a primer capable of amplifying a nucleic acid sequence derived from a Mycoplasma (a primer specifically configured or selected to amplify a nucleic acid sequence derived from a Mycoplasma), in which the specimen is a biotechnology-related product or a part thereof.

[2] The method described in [1], in which the specimen is a regenerative medicine-related product or a part thereof.

[3] The method described in [1] or [2], in which the step of performing amplification of the nucleic acid is performed by a polymerase chain reaction (PCR) method.

[4] The method described in any one of [1] to [3], further including (c) a step of performing electrophoresis of an amplified product obtained from the step (b).

[5] The method described in any one of [1] to [4], in which the method includes performing anaerobic cultivation in the step (a).

[6] The method described in any one of [1] to [4], in which in the step (a), two or more specimens are each cultured in an aerobic or anaerobic condition.

[7] The method described in any one of [1] to [6], in which an amount of the specimen cultured in the step (a) is 10 μL to 10 mL.

[8] The method described in any one of [1] to [7], in which in the step (a), cultivation is performed for 3 to 14 days.

[9] The method described in any one of [1] to [8], further including removing impurities before the nucleic acid is extracted in the step (b).

[10] The method described in any one of [1] to [9], in which removing of impurities is performed by collecting the specimen with a filter.

[11] The method described in any one of [1] to [10], in which a molecular weight cutoff of a filter is 1000 kDa or less or a pore size of a filter is 0.22 μm or less.

[12] The method described in any one of [1] to [11], in which the regenerative medicine-related product includes at least one selected from a cell subjected to processing, a cell to be provided to processing, a reagent to be used in processing or testing, a culture medium for culturing a cell, a cell washing solution for washing a cell, and a preservation liquid for preserving a regenerative medicine product.

[13] According to another aspect, a method of managing quality of a biotechnology-related product involves: a step of testing presence or absence of a Mycoplasma in a specimen by the method described in any one of [1] to [12].

[14] A kit for use in the method described in any one of [1] to [12], the kit including: a culture medium for culturing a Mycoplasma; and a primer capable of amplifying a nucleic acid sequence derived from a Mycoplasma.

[15] The kit described in [14], further including a filter for removing impurities.

[I] A method for testing presence or absence of a Mycoplasma in a specimen, the method including:
(i) a step of removing impurities from a composition containing a specimen; and
(ii) a step of extracting a nucleic acid from the composition containing a specimen from which impurities have been removed and performing amplification of the nucleic acid using a primer capable of amplifying a nucleic acid sequence derived from a Mycoplasma.

[II] The method described in [I], in which the step of performing amplification of the nucleic acid is performed by a polymerase chain reaction (PCR) method.

[III] The method described in [I] or [II], further including (iii) a step of performing electrophoresis of an amplified product obtained from the step (ii).

[IV] The method described in any one of [I] to [III], in which in the step (i), the step of removing impurities is performed by collecting the specimen with a filter.

[V] The method described in any one of [I] to [IV], in which a molecular weight cutoff of a filter is 1000 kDa or less or a pore size of a filter is 0.22 μm or less.

[VI] The method described in any one of [I] to [V], in which the method includes culturing the specimen before the step (ii).

[VII] The method described in any one of [I] to [VI], in which the method includes culturing the specimen before the step (i).

[VIII] The method described in any one of [I] to [VII], in which the specimen is a biotechnology-related product or a part thereof.

[IX] The method described in any one of [I] to [VIII], in which the specimen is a regenerative medicine-related product or a part thereof.

[X] The method described in any one of [I] to [IX], in which the regenerative medicine-related product includes at least one selected from a cell subjected to processing, a cell to be provided to processing, a reagent to be used in processing or testing, a culture medium for culturing a cell, a cell washing solution for washing a cell, and a preservation liquid for preserving a regenerative medicine product.

[XI] According to another aspect, a method of managing quality of a biotechnology-related product involves: a step of testing presence or absence of a Mycoplasma in a specimen by the method described in any one of [I] to [X].

[XII] A kit for use in the method described in any one of [I] to [X], the kit including: a primer capable of amplifying a nucleic acid sequence derived from a Mycoplasma; and a filter for removing impurities.

According to another aspect, a method for testing for a presence or absence of a Mycoplasma in a specimen comprises: obtaining a cultured regenerative medicine-related product or part of the regenerative medicine-related product, with the regenerative medicine-related product or part of the regenerative medicine-related product that is cultured being in an amount of 950 μL or less; extracting a nucleic acid of a Mycoplasma from the cultured regenerative medicine-related product or part of the regenerative medicine-related product; and amplifying the nucleic acid of the Mycoplasma by a polymerase chain reaction method to amplify a nucleic acid sequence derived from the Mycoplasma.

In an aspect of the disclosure here, it is possible to detect a Mycoplasma with sensitivity of 10 cfu/mL or less in a short time even from a small amount of a specimen. Only a relatively short period of time is required for a test and only a small amount of specimen is needed. Thus, a method described below is especially useful in the field of biotechnology and particularly in the field of regenerative medicine. Further, by removing impurities before amplifying a nucleic acid, a test with reduced false positives and thus high accuracy can be performed.

According to another aspect, a method for preparing a sample for testing for the presence or absence of a Mycoplasma in a specimen comprises culturing a sample from the specimen in a culture medium containing arginine and glucose for Mycoplasma proliferation, wherein the culturing comprises aerobic culturing of a first sample from the specimen and anaerobic culturing of a second sample from the specimen which is different from the first sample, and providing the first and second samples for detecting seven species of Mycoplasmas to be applied for nucleic acid amplification.

In accordance with another aspect, a method for preparing a sample for testing for the presence or absence of a Mycoplasma in a specimen comprises culturing a sample of 950 μL or less from the specimen in the culture medium containing arginine and glucose for Mycoplasma proliferation, and providing the first and second samples for detecting seven species of Mycoplasmas applied for nucleic acid amplification.

According to a further aspect, a method for testing for a presence or absence of a Mycoplasma in a specimen comprises: culturing a specimen to obtain a cultured specimen, the specimen being a biotechnology-related product or a part of a biotechnology-related product; extracting a nucleic acid from the cultured specimen; and performing amplification of the nucleic acid using a primer that is specifically selected to amplify a nucleic acid sequence derived from a Mycoplasma.

### BRIEF DESCRIPTION OF THE DRAWINGS

FIGS. 1(A) and 1(B) are electrophoretic profiles of a PCR product. In (A), M shows a 100 bp ladder, Lane 1 shows the result when PCR is performed without addition of a Mycoplasma, and Lane 2 shows a positive control (the result when PCR is performed with addition of a Mycoplasma (Acholeplasma laidlawii)). In (B), M shows a 100 bp ladder, Lanes 1 to 3 show the result when PCR is performed without addition of a Mycoplasma after collection with a filter, and Lanes 4 to 6 show a positive control (the result when PCR is performed with addition of a Mycoplasma (Acholeplasma laidlawii) after collection with a filter).

FIGS. 2(A) and 2(B) are graphs showing proliferation of a Mycoplasma in a cellular specimen. FIG. 2(A) shows a result of aerobic cultivation and FIG. 2(B) shows a result of anaerobic cultivation. The vertical axis shows a concentration (cfu/mL, logarithmic expression) of a Mycoplasma and the horizontal axis shows cultivation days (day).

FIGS. 3(A) and 3(B) are graphs showing proliferation of a Mycoplasma in a culture medium specimen. FIG. 3(A) shows a result of aerobic cultivation and FIG. 3(B) shows a result of anaerobic cultivation. The vertical axis shows a concentration (cfu/mL, logarithmic expression) of a Mycoplasma and the horizontal axis shows cultivation days (day).

FIGS. 4(A) and 4(B) are graphs showing proliferation of a Mycoplasma in a washing solution specimen. FIG. 4(A) shows a result of aerobic cultivation and FIG. 4(B) shows a result of anaerobic cultivation. The vertical axis shows a concentration (cfu/mL, logarithmic expression) of a Mycoplasma and the horizontal axis shows cultivation days (day).

FIG. 5 shows proliferation and a detection limit of a Mycoplasma in a cellular specimen. The vertical axis shows a concentration (cfu/m L, logarithmic expression) of a Mycoplasma. In the drawing, black represents a concentration of a Mycoplasma after cultivation for 7 days and gray represents detection sensitivity.

FIG. 6 shows proliferation and a detection limit of a Mycoplasma in a culture medium specimen. The vertical axis shows a concentration (cfu/mL, logarithmic expression) of a Mycoplasma. In the drawing, black represents a concentration of a Mycoplasma after cultivation for 7 days and gray represents detection sensitivity.

FIG. 7 shows proliferation and a detection limit of a Mycoplasma in a washing solution specimen. The vertical axis shows a concentration (cfu/m L, logarithmic expression) of a Mycoplasma. In the drawing, black represents a concentration of a Mycoplasma after cultivation for 7 days and gray represents detection sensitivity.

FIG. 8 is an electrophoretic profile of a PCR product. M shows a 100 bp ladder, Lane 1 shows a negative control (the result when PCR is performed with respect to water), Lanes 2 to 4 show a negative control (the result when PCR is performed by aerobic cultivation using PBS instead of a specimen), Lanes 5 to 7 show the result when PCR is performed by aerobic cultivation of a specimen, Lanes 8 to 10 show a positive control (the result when PCR is performed by aerobic cultivation with addition of a Mycoplasma diluent (Mycoplasma orale) to a specimen), Lanes 11 to 13 show a negative control (the result when PCR is performed by anaerobic cultivation using PBS instead of a specimen), Lanes 14 to 16 show the result when PCR is performed by anaerobic cultivation of a specimen, and Lanes 17 to 19 show a positive control (the result when PCR is performed by anaerobic cultivation with addition of a Mycoplasma diluent (Mycoplasma salivarium) to a specimen).

### DETAILED DESCRIPTION

Set forth below with reference to the accompanying drawings is a detailed description of embodiments of method for testing the presence or absence of a Mycoplasma representing examples of the method disclosed here.

Unless defined otherwise in this specification, all technical terms and scientific terms used have the same meanings as usually understood by those skilled in the art. All patents, applications, and other publications or information referred to in this specification are incorporated in this specification by reference in their entireties. Further, if any contradiction arises between publications referred to in this specification and descriptions in this specification, the descriptions in this specification supersede.

A method for testing the presence or absence of a Mycoplasma in a specimen includes: a step of extracting a nucleic acid from a specimen and performing amplification of the nucleic acid using a primer capable of amplifying a nucleic acid sequence derived from a Mycoplasma.

As used here, the “specimen” is not particularly limited and may be any target in which contamination by Mycoplasma is desired to be tested (e.g., a target in which there is a concern of Mycoplasma contamination, a part thereof, or the like). Non-limiting examples of targets include biomedical materials of any biological origin (for example, tissues, cells, biologic fluids, and the like) and products produced using biomedical materials as raw materials (for example, some of organs, fusion cells, pharmaceutical products of biological origin, and the like). Without limitation, specific examples include animal-derived biomedical materials and products produced using these materials as raw materials, and those which are provided for tests or production performed using animal-derived biomedical materials. Examples of the animal-derived biomedical materials and products produced using these materials as raw materials include, but are not limited to, cells, tissues, blood (whole blood or some of whole blood, for example, including serum, blood plasma, and the like), placenta, urine, and antibodies of animals, biological preparations (for example, proteins, polypeptide, derivatives thereof, and pharmaceutical products, antibody pharmaceutical products, vaccines, antitoxins, blood preparations, and the like which use proteins, polypeptide, or derivatives thereof as constituents), and regenerative medicine products. Examples of those which are provided for tests or production performed using animal-derived biomedical materials include, but are not limited to, tools which are provided for tests or production performed using animal-derived biomedical materials (for example, cell culture vessels and the like), reagents, serum, antibodies, culture media, and washing solutions.

Further, the “specimen” as used here may be those which are produced as a biotechnology-related product or a regenerative medicine-related product.

As used here, the “biotechnology-related product” includes a biological preparation, a regenerative medicine product, or a product and the like which are used in production thereof.

As used here, the “biological preparation” includes, for example, proteins, polypeptide, derivatives thereof, and pharmaceutical products, antibody pharmaceutical products, vaccines, antitoxins, blood preparations, and the like which use proteins, polypeptide, or derivatives thereof as constituents.

As used here, a product used in production of a biological preparation includes a cell producing a recombination protein or a polypeptide, blood (whole blood or some of whole blood, for example, including serum, blood plasma, and the like), placenta, urine, a culture medium for culturing a cell, a cell washing solution for washing a cell, a preservation liquid for preserving a biological preparation, a reagent to be used in processing or testing (for example, a buffer solution, a detection reagent, serum, a growth factor, an yeast extract, or the like), and the like.

Examples of the cell producing a recombination protein or a polypeptide include E. coli, yeast, insect cells, plant cells, hybridoma, animal cells (for example, CHO cells, Sp2/0 cells, NSO cells, and the like), and human cells. In an aspect of methods described herein, the product used in production of a biological preparation can include a vaccine, but the vaccine can also be excluded from the product used in production of a biological preparation.

As used here, the “regenerative medicine-related product” includes a regenerative medicine product or a product and the like which are used in production of the regenerative medicine product.

As used here, the “regenerative medicine product” is (1) a product which is obtained by subjecting cells of humans or animals to processing such as cultivation, and which (a) reconstructs/restores/forms a structure/function of a body or (b) is used for medical treatment/prevention of diseases, or is (2) a product which is introduced into cells of a human and then used for the purpose of gene therapy. The “regenerative medicine product” includes a cell used in medical treatment as a body, and for example, includes a cell subjected to processing and a cell to be provided to processing. Specifically, non-limiting examples thereof include human (allogeneic) bone marrow-derived mesenchymal stem cells used in medical treatment of acute graft-versus-host disease (acute GVHD) after hematopoietic stem cell transplantation, human (autologous) skeletal muscle-derived cell sheets to be transplanted in medical treatment of serious heart failures, and cells to be used in cancer immunotherapy (such as dendritic cells, NK cells, and T lymphocytes).

As used here, a product used in production of the regenerative medicine product includes a reagent to be used in processing or testing (for example, a buffer solution, a detection reagent, serum, a growth factor, an yeast extract, or the like) a culture medium for culturing a cell, a cell washing solution for washing a cell, a preservation liquid for preserving a regenerative medicine product, and the like.

As used here, the cell subjected to processing and the cell to be provided to processing are animal cells, preferably mammal cells, and more preferably human cells.

Non-limiting examples of the cell subjected to processing and the cell to be provided to processing include somatic cells (for example, myocardial cells, fibroblasts, epithelial cells, endothelial cells, hepatic cells, pancreatic cells, renal cells, adrenal cells, periodontal ligament cells, gingival cells, periosteal cells, skin cells, synovial cells, cartilage cells, dendritic cells, lymphocytes (natural killer (NK) cells, T lymphocytes, and B lymphocytes), and the like), and stem cells (for example, tissue stem cells such as myoblasts and cardiac stem cells, embryonic stem cells (ES) cells), pluripotent stem cells such as induced pluripotent stem (iPS) cells, mesenchymal stem cells, and the like). The somatic cells may be stem cells, in particular, cells differentiated from iPS cells, and specific examples thereof include iPS-derived myocardial cells, iPS-derived dendritic cells, and iPS-derived lymphocytes (NK cells, T lymphocytes, and B lymphocytes). These cells may be modified by gene introduction, and specific examples of such cells include a chimeric antigen receptor-T cell (CAR-T) and a T cell receptor-T cell (TCR-T).

The cell subjected to processing refers to a cell obtainable after being subjected to some kind of processing, and the cell to be provided to processing refers to a cell to be prepared before being subjected to processing. The processing is not limited, and may be any operation applied to cells, and for example, may be processing for treatment of diseases. Specific examples of such cells include cells cultured in a liquid, cells cultured in the environment, for example, in a gel, cell cultures cultured and then three-dimensionally constructed via cellular adhesion, sheet-shaped cell cultures, cell suspensions in which a cell density is adjusted, cells cultured and grown, cells cultured and provided to freezing, cells melted from a freeze state, cells in which the number of cells or density thereof is adjusted, and cells to be set for administration in an administration device.

Diseases to be treated by such cells are not limited, and examples thereof include cardiac diseases, lung diseases, liver diseases, pancreatic disorders, kidney diseases, large intestine diseases, small intestine diseases, spinal cord diseases, central nervous system diseases, bone diseases, eye diseases, and skin diseases. Cancers (malignant neoplasm) such as blood cancer and solid cancer are included in diseases. In a case where cells to be used are myoblasts or (iPS-derived) myocardial cells, examples of diseases include cardiac infarction (including chronic cardiac failure accompanied by cardiac infarction), dilated cardiomyopathy, ischemic cardiomyopathy, and cardiac diseases (for example, cardiac failure, particularly chronic cardiac failure) accompanied by systolic functional disorder (for example, left systolic functional disorder).

In an embodiment, the regenerative medicine-related product includes at least one cell which either has been or will be subjected to processing or provided to processing, a reagent to be used in processing or testing, a culture medium for culturing a cell, a cell washing solution for washing a cell, and a preservation liquid for preserving a regenerative medicine product. Specific examples include, but are not limited to, a sheet-shaped cell culture, a cell for producing a sheet-shaped cell culture (for example, a cryopreserved cell or the like), a sheet preparation culture medium, and a cell washing solution.

In an embodiment, the regenerative medicine-related product includes at least one selected from a cell subjected to processing and/or a cell to be provided to processing.

In an embodiment, the regenerative medicine-related product includes at least one selected from myoblasts and iPS-derived myocardial cells.

In an embodiment, the regenerative medicine-related product includes at least one mesenchymal stem cell and/or fibroblast.

In an embodiment, the regenerative medicine-related product includes at least one dendritic cell, NK cell, T lymphocyte, B lymphocyte, CAR-T, and/or TCR-T.

As used here, the sheet-shaped cell culture refers to cells linked together into the shape of a sheet. Cells may be linked together directly (including cellular adhesion linked via cellular elements, such as adhesion molecules) and/or linked together via an intervening substance. The intervening substance is not particularly limited as long as it is a substance having at least an ability to physically (mechanically) link cells together, and examples thereof include extracellular matrices. The intervening substance may be a substance derived from cells, in particular, a substance derived from cells constituting the cell culture. Although the cells are linked at least physically (mechanically), the cells may be further linked functionally, for example, chemically or electrically. The sheet-shaped cell culture may be a culture configured with a single cell layer (single layer), or a culture configured with two or more cell layers (stacked (multilayer) architecture, for example, two layers, three layers, four layers, five layers, six layers, and the like).

The sheet-shaped cell culture is used in medical treatment of humans or animals, and cells constituting the sheet-shaped cell culture may be animal cells, preferably mammal cells, and more preferably human cells.

The cells constituting the sheet-shaped cell culture are not particularly limited as long as they can form the sheet-shaped cell culture, and for example, include adhesion cells (adherent cells). Examples of suitable adhesion cells include, but are not limited to, adherent somatic cells (for example, myocardial cells, fibroblasts, epithelial cells, endothelial cells, hepatic cells, pancreatic cells, renal cells, adrenal cells, periodontal ligament cells, gingival cells, periosteal cells, skin cells, synovial cells, cartilage cells, and the like) and stem cells (for example, tissue stem cells such as myoblasts and cardiac stem cells, embryonic stem cells, pluripotent stem cells such as induced pluripotent stem (iPS) cells, mesenchymal stem cells, and the like). The somatic cells may be stem cells, such as cells differentiated from iPS cells.

Further, non-limiting examples of the cells constituting the sheet-shaped cell culture include myoblasts (for example, skeletal myoblasts and the like), mesenchymal stem cells (for example, bone marrow, adipose tissue, peripheral blood, skin, hair root, muscular tissue, endometrium, placenta, cord blood-derived cells, and the like), myocardial cells, fibroblasts, cardiac stem cells, embryonic stem cells, iPS cells, synovial cells, cartilage cells, epithelium cells (for example, oral mucosaepithelium cells, retinal pigment epithelial cells, nasal epithelial cells, and the like), endothelial cells (for example, vascular endothelial cells and the like), hepatocytes (for example, hepatic parenchymal cells and the like), pancreatic cells (for example, islet cells and the like), renal cells, adrenal gland cells, human periodontal ligament cells, gingiva cells, periosteum cells, and skin cells. The sheet-shaped cell culture is configured by these cells, and specific examples thereof include a skeletal myoblast sheet, an iPS-derived myocardial cell sheet, a mesenchymal stem cell sheet, and a fibroblast sheet.

The Mycoplasma is not particularly limited as long as it is a bacterium classified in the class Mollicutes. Examples include, but are not limited to, bacteria belonging to the genus Mycoplasma in the family Mycoplasmataceae, the genus Acholeplasma in the family Acholeplasmataceae, the genus Spiroplasma in the family Spiroplasmataceae, and the like. More specific examples thereof include Mycoplasma arginini, Mycoplasma salivarium, Mycoplasma pneumoniae, Mycoplasma fermentans, Mycoplasma orale, Mycoplasma hyorhinis, Mycoplasma synoviae, Acholeplasma laidlawii, and Spiroplasma citri. In this specification, Mycoplasma arginini is also referred to as Ma, Mycoplasma salivarium is also referred to as Ms, Mycoplasma pneumoniae is also referred to as Mp, Mycoplasma fermentans is also referred to as Mf, Mycoplasma orale is also referred to as Mo, Mycoplasma hyorhinis is also referred to as Mh, and Acholeplasma laidlawii is also referred to as Al.

It is sufficient that the amount of a specimen is an amount necessary for amplifying a Mycoplasma to an amount beyond the detection sensitivity of this method, and it is possible for those having ordinary knowledge in the relevant technical field to set an appropriate amount. Non-limiting examples thereof include 10 μL, 15 μL, 20 μL, 25 μL, 30 μL, 35 μL, 40 μL, 45 μL, 50 μL, 55 μL, 60 μL, 65 μL, 70 μL, 75 μL, 80 μL, 85 μL, 90 μL, 95 μL, 100 μL, 110 μL, 120 μL, 130 μL, 140 μL, 150 μL, 160 μL, 170 μL, 180 μL, 190 μL, 200 μL, 250 μL, 300 μL, 350 μL, 400 μL, 450 μL, 500 μL, 550 μL, 600 μL, 650 μL, 700 μL, 750 μL, 800 μL, 850 μL, 900 μL, 950 μL, 1 mL, 2 mL, 3 mL, 4 mL, 5 mL, 6 mL, 7 mL, 8 mL, 9 mL, and 10 mL.

The lower limit of the amount of a specimen is not particularly limited as long as it is an amount necessary for amplifying a Mycoplasma to an amount beyond the detection sensitivity of this method. Non-limiting examples thereof include 10 μL or more, 15 μL or more, 20 μL or more, 25 μL or more, 30 μL or more, 35 μL or more, 40 μL or more, 45 μL or more, 50 μL or more, 55 μL or more, 60 μL or more, 65 μL or more, 70 μL or more, 75 μL or more, 80 μL or more, 85 μL or more, 90 μL or more, 95 μL or more, and 100 μL or more. In certain embodiments, the amount of a specimen is 100 μL.

The upper limit of the amount of a specimen is not particularly limited, but in the case of the method described here, in a regenerative medicine-related product, among amounts in which the detection sensitivity of this method can be achieved, the amount is preferably an amount as small as possible. Non-limiting examples thereof include 10 mL or less, 9 mL or less, 8 mL or less, 7 mL or less, 6 mL or less, 5 mL or less, 4 mL or less, 3 mL or less, 2 mL or less, 1 mL or less, 950 μL or less, 900 μL or less, 850 μL or less, 800 μL or less, 750 μL or less, 700 μL or less, 650 μL or less, 600 μL or less, 550 μL or less, 500 μL or less, 450 μL or less, 400 μL or less, 350 μL or less, 300 μL or less, 250 μL or less, 200 μL or less, 150 μL or less, and 100 μL or less.

As for the range of the amount of a specimen, combinations of the upper limits and the lower limits exemplified above are mentioned. That is, examples thereof include 10 μL to 10 mL, 10 μL to 9 mL, 10 μL to 8 mL, 10 μL to 7 mL, 10 μL to 6 mL, 10 μL to 5 mL, 10 μL to 4 mL, 10 μL to 3 mL, 10 μL to 2 mL, 10 μL to 1 mL, 10 to 900 μL, 10 to 800 μL, 10 to 700 μL, 10 to 600 μL, 10 to 500 μL, 10 to 400 μL, 10 to 300 μL, 10 to 200 μL, 10 to 100 μL, 100 μL to 10 mL, 100 μL to 9 mL, 100 μL to 8 mL, 100 μL to 7 mL, 100 μL to 6 mL, 100 μL to 5 mL, 100 μL to 4 mL, 100 μL to 3 mL, 100 μL to 2 mL, 100 μL to 1 mL, 100 to 900 μL, 100 to 800 μL, 100 to 700 μL, 100 to 600 μL, 100 to 500 μL, 100 to 400 μL, 100 to 300 μL, and 100 to 200 μL. The range thereof is particularly preferably 100 to 1000 μL and further preferably 100 to 500 μL.

The nucleic acid is DNA or RNA, and is preferably DNA.

The method of extracting a nucleic acid from a specimen is not particularly limited as long as it can extract a nucleic acid of a Mycoplasma, and it is possible for those skilled in the art to appropriately select an optimal method. For example, phenol/chloroform extraction, an extraction method using a carrier, and the like may be employed. Extraction of a nucleic acid may be performed using a commercially available kit for nucleic acid extraction.

The method of amplifying a nucleic acid is not particularly limited as long as it can amplify a nucleic acid sequence derived from a Mycoplasma, and it is possible for those skilled in the art to appropriately select an optimal method.

Examples of the method of amplifying a nucleic acid include a polymerase chain reaction (PCR) method and a loop-mediated isothermal amplification (LAMP) method.

The step of performing amplification of the nucleic acid is performed by a polymerase chain reaction (PCR) method. In the PCR method, a primer capable of amplifying a nucleic acid sequence derived from a Mycoplasma, polymerase, a buffer, PCR conditions (temperature, time, the number of cycles, and the like), and the like can be appropriately selected.

PCR may be, for example, regular PCR, real-time PCR, Nested PCR, reverse transcription PCR, or the like. Regular PCR is preferred.

As described above, as for the primer used here, any primers may be used as long as they can amplify a nucleic acid sequence derived from a Mycoplasma. Non-limiting examples of the primer capable of amplifying a nucleic acid sequence derived from a Mycoplasma include the following sequences:

Forward:
(SEQ ID NO: 1)
5′-GGCGAATGGGTGAGTAACACG-3′,
and
Reverse:
(SEQ ID NO: 2)
5′-CGGATAACGCTTGCGACCTATG-3′.

A method for testing presence or absence of a Mycoplasma in a specimen described here comprises:
(a) a step of culturing a specimen; and
(b) a step of extracting a nucleic acid from the cultured specimen and performing amplification of the nucleic acid using a primer capable of amplifying a nucleic acid sequence derived from a Mycoplasma ,
   in which the specimen is a biotechnology-related product or a part thereof.

In an embodiment, the specimen is a regenerative medicine-related product or a part thereof.

In an embodiment, the regenerative medicine-related product includes at least one cell subjected to processing or cell to be provided to processing, a reagent to be used in processing or testing, a culture medium for culturing a cell, a cell washing solution for washing a cell, and a preservation liquid for preserving a regenerative medicine product. Specific examples thereof include a sheet-shaped cell culture, a cell for producing a sheet-shaped cell culture (for example, a cryopreserved cell or the like), a sheet preparation culture medium, and a cell washing solution.

In an embodiment, this method further includes (c) performing electrophoresis of an amplified PCR product obtained from (b).

The electrophoresis of the amplified product is not particularly limited as long as it is a method with which the amplified nucleic acid can be detected, and it is possible for those skilled in the art to appropriately select an optimal method. For example, as for a method for preparing a gel such as agarose gel, selection of a buffer, and a method of checking a band, it is possible for those skilled in the art to appropriately select an optimal method. Examples of the method of checking a band include staining of a nucleic acid with ethidium bromide, SYBR (registered trademark) Green, or the like and a southern blotting method.

In the methods disclosed here, conditions of the cultivation are not particularly limited as long as a method can amplify a Mycoplasma, and it is possible for those skilled in the art to appropriately select an optimal method. For example, types and composition of a culture medium, a cultivation temperature, an atmosphere, a CO₂ concentration, and the like can also be appropriately selected.

In the methods disclosed here, cultivation may be performed by aerobic cultivation or anaerobic cultivation and can be appropriately selected according to types of a Mycoplasma to be tested. For example, as for five kinds of Mycoplasma arginini, Mycoplasma pneumoniae, Mycoplasma fermentans, Mycoplasma orale, and Acholeplasma laidlawii, these Mycoplasmas can be amplified under any of an aerobic condition and an anaerobic condition; however, since Mycoplasma salivarium can be amplified only under an anaerobic condition and Mycoplasma hyorhinis can be amplified only under an aerobic condition, six kinds other than Mycoplasma salivarium may be cultured under an aerobic condition and Mycoplasma salivarium may be cultured under an anaerobic condition. In order to perform testing of all seven kinds, two or more specimens can be each cultured in an aerobic or anaerobic condition.

In an embodiment, this method includes performing anaerobic cultivation in (a).

In an embodiment, in this method, two or more specimens are each cultured in an aerobic or anaerobic condition.

In an embodiment, an amount of a specimen to be cultured in (a) is 10 μL to 10 mL.

In an embodiment, an amount of a specimen to be cultured in (a) is 100 μL to 1 mL.

In an embodiment, an amount of a specimen to be cultured in (a) is 100 μL to 500 μL.

The period of time for cultivation may be a period which is sufficient for amplifying a Mycoplasma to an amount beyond the detection sensitivity obtained by this method, and can be arbitrarily set. Non-limiting examples of the period of time for cultivation include 1 day, 2 days, 3 days, 4 days, 5 days, 6 days, 7 days, 8 days, 9 days, 10 days, 11 days, 12 days, 13 days, and 14 days. Non-limiting examples of the range of the period of time for cultivation include 1 day to 14 days, 1 day to 7 days, 3 days to 14 days, 3 days to 7 days, and 6 days to 10 days. The time period range is preferably 3 days to 14 days and more preferably 6 days to 10 days.

In an embodiment, cultivation is performed for 6 days to 10 days.

Further, in an embodiment, cultivation is performed for 3 days to 7 days.

In an embodiment, the method further includes removing impurities before the nucleic acid is extracted in (b).

The impurities are, for example, substances other than bacteria of Mycoplasmas, and specifically, are nucleic acids, proteins, and the like, and are preferably nucleic acid fragments of Mycoplasma s.

As for removal of the impurities, a method with which impurities decrease as compared with the case before removal is sufficient. Examples of the method of removing impurities include a method of filtering or collecting a specimen by a filter and a method of performing harvest by centrifugal separation.

The impurities may pass through a filter so as to be removed or may remain on a filter without passing through a filter so as to be removed. Preferably, the impurities pass through a filter so as to be removed.

In an embodiment, removal of the impurities is performed by collecting the specimen with a filter.

As for the filter for collection, a commercially available filter can be used. The pore size of the filter is, for example, 0.22 μm, 0.1 μm, 0.01 μm, and 0.005 μm. For example, in the case of a filter having a pore size of 0.1 μm, a substance having a size of about 0.1 μm, for example, a virus or the like can be trapped. The filter may be a filter which is designed to exclude impurities having a specific molecular weight, and for example, the molecular weight cutoff (MWCO) of the filter is 1000 kDa, 100 kDa, and 50 kDa. For example, the filter having a molecular weight cutoff of 100 kDa is a filter which can trap a substance having a size of about 100 kDa. The molecular weight cutoff of the filter is 50 to 1000 kDa and preferably 50 to 100 kDa.

Impurities may be smaller than the molecular weight cutoff of the filter and be allowed to pass through the filter so as to be removed, or may be larger than the molecular weight cutoff of the filter and remain on the filter without being allowed to pass through the filter so as to be removed. Preferably, impurities are smaller than the molecular weight cutoff of the filter and pass through the filter.

A molecular weight cutoff of a filter is 1000 kDa or less or a pore size of a filter is 0.22 μm or less.

In an embodiment, a molecular weight cutoff of a filter is 100 kDa or less.

An aspect of the disclosure here relates to a method of managing quality of a biotechnology-related product, the method including testing presence or absence of a Mycoplasma in a specimen by the method described herein.

An aspect of the disclosure relates to a method of managing quality of a regenerative medicine-related product, the method including testing presence or absence of a Mycoplasma in a specimen by the method described herein.

A method disclosed here for testing the presence or absence of a Mycoplasma in a specimen involves:
(i) a step of removing impurities from a composition containing a specimen; and
(ii) a step of extracting a nucleic acid from the composition containing a specimen from which impurities have been removed and performing amplification of the nucleic acid using a primer capable of amplifying a nucleic acid sequence derived from a Mycoplasma.

The composition containing a specimen is, for example, a composition containing a culture medium, a reagent, or the like, which is used for testing presence or absence of a Mycoplasma, in addition to a specimen.

Impurities are, for example, substances other than bacteria of Mycoplasmas, and specifically, are nucleic acids, proteins, and the like, and are preferably nucleic acid fragments of Mycoplasma s.

As for removal of the impurities, a method with which impurities decrease as compared with the case before removal is sufficient. Examples of the method of removing impurities include a method of filtering or collecting a specimen by a filter and a method of performing harvest by centrifugal separation.

The impurities may pass through a filter so as to be removed or may remain on a filter without passing through a filter so as to be removed. Preferably, the impurities pass through a filter so as to be removed.

In the step (i), the step of removing impurities is performed by collecting the specimen with a filter.

As for the filter for collection, a commercially available filter can be used. The pore size of the filter is, for example, 0.22 μm, 0.1 μm, 0.01 μm, and 0.005 μm. For example, in the case of a filter having a pore size of 0.1 μm, a substance having a size of about 0.1 μm, for example, a virus or the like can be trapped. The filter may be a filter which is designed to exclude impurities having a specific molecular weight, and for example, the molecular weight cutoff (MWCO) of the filter is 1000 kDa, 100 kDa, and 50 kDa. For example and without limitation, the filter having a molecular weight cutoff of 100 kDa may be a filter which can trap a substance having a size of about 100 kDa. In certain embodiments, the molecular weight cutoff of the filter is 50 to 1000 kDa and preferably 50 to 100 kDa.

Impurities may be smaller than the molecular weight cutoff of the filter and be allowed to pass through the filter so as to be removed, or may be larger than the molecular weight cutoff of the filter and remain on the filter without being allowed to pass through the filter so as to be removed. Preferably, impurities are smaller than the molecular weight cutoff of the filter and pass through the filter.

A molecular weight cutoff of a filter is 1000 kDa or less or a pore size of a filter is 0.22 μm or less.

A molecular weight cutoff of a filter is 100 kDa or less.

This method further includes (iii) a step of performing electrophoresis of an amplified product obtained from the step (ii).

The electrophoresis of the amplified product is not particularly limited as long as it is a method with which the amplified nucleic acid can be detected, and it is possible for those skilled in the art to appropriately select an optimal method. For example, as for a method for preparing a gel such as agarose gel, selection of a buffer, and a method of checking a band, it is possible for those skilled in the art to appropriately select an optimal method. Examples of the method of checking a band include staining of a nucleic acid with ethidium bromide, SYBR (registered trademark) Green, or the like and a southern blotting method.

Conditions of the cultivation are not particularly limited as long as a method can amplify a Mycoplasma, and it is possible for those skilled in the art to appropriately select an optimal method. For example, types and composition of a culture medium, a cultivation temperature, an atmosphere, a CO₂ concentration, and the like can also be appropriately selected.

Cultivation may be performed by aerobic cultivation or anaerobic cultivation and can be appropriately selected according to types of a Mycoplasma to be tested.

For example, as for five kinds of Mycoplasma arginini, Mycoplasma pneumoniae, Mycoplasma fermentans, Mycoplasma orale, and Acholeplasma laidlawii, these Mycoplasmas can be amplified under any of an aerobic condition and an anaerobic condition; however, since Mycoplasma salivarium can be amplified only under an anaerobic condition and Mycoplasma hyorhinis can be amplified only under an aerobic condition, six kinds other than Mycoplasma salivarium may be cultured under an aerobic condition and Mycoplasma salivarium may be cultured under an anaerobic condition. In order to perform testing of all seven kinds, two or more specimens can be each cultured in an aerobic or anaerobic condition.

In an embodiment, the method includes culturing the specimen before (ii).

In an embodiment, the method includes culturing the specimen before (i).

The period of time for cultivation may be a period which is sufficient for amplifying a Mycoplasma to an amount beyond the detection sensitivity obtained by this method, and can be arbitrarily set. Non-limiting examples of the period of time for cultivation include 1 day, 2 days, 3 days, 4 days, 5 days, 6 days, 7 days, 8 days, 9 days, 10 days, 11 days, 12 days, 13 days, and 14 days. Non-limiting examples of the range of the period of time for cultivation include 1 day to 14 days, 1 day to 7 days, 3 days to 14 days, 3 days to 7 days, and 6 days to 10 days. The time period range is preferably 3 days to 14 days and more preferably 6 days to 10 days.

In an embodiment, the specimen is a biotechnology-related product or a part thereof.

In an embodiment, the specimen is a regenerative medicine-related product or a part thereof.

In an embodiment, the regenerative medicine-related product includes at least one selected from a cell subjected to processing, a cell to be provided to processing, a reagent to be used in processing or testing, a culture medium for culturing a cell, a cell washing solution for washing a cell, and a preservation liquid for preserving a regenerative medicine product. Specific examples thereof include a sheet-shaped cell culture, a cell for producing a sheet-shaped cell culture (for example, a cryopreserved cell or the like), a sheet preparation culture medium, and a cell washing solution.

An aspect of the disclosure here involves a method of managing quality of a biotechnology-related product, the method including a step of testing presence or absence of a Mycoplasma in a specimen by a method described herein.

An aspect of the disclosure here involves a method of managing quality of a regenerative medicine-related product, the method including a step of testing presence or absence of a Mycoplasma in a specimen by a method described herein.

Another aspect of the disclosure here involves a kit for use in the method for testing for the presence or absence of a Mycoplasma, the kit including a primer capable of amplifying a nucleic acid sequence derived from a Mycoplasma.

In an embodiment, the kit includes, in addition to the primer capable of amplifying a nucleic acid sequence derived from a Mycoplasma, a culture medium for culturing a Mycoplasma and/or a filter for removing impurities.

In an embodiment, the kit may further include, for example, a tool for culturing a Mycoplasma, a reagent or tool used in extraction of a nucleic acid, a reagent used in amplification of a nucleic acid, other tools, and instructions relating to a usage method.

Examples of the tool for culturing a Mycoplasma include a plate and a tube. Examples of the tool or reagent used in extraction of a nucleic acid include a reagent containing phenol, chloroform, isopropanol, ethanol, PEG, or the like, a column filled with silica beads or a silica membrane, and an anion-exchange column. Examples of the reagent used in amplification of a nucleic acid include polymerase, dNTP, and a buffer. Examples of the other tools include a pipette, a dropper, tweezers, and a tube. Examples of the instructions relating to a usage method include instructions for use and a medium on which information indicating a production method or a usage method, such as a flexible disk, a CD, a DVD, a Blu-ray Disc, a memory card, or an USB memory.

In an embodiment, the kit includes a culture medium for culturing a Mycoplasma and a primer capable of amplifying a nucleic acid sequence derived from a Mycoplasma.

In an embodiment, the kit includes a primer capable of amplifying a nucleic acid sequence derived from a Mycoplasma and a filter for removing impurities.

In an embodiment, the kit includes a culture medium for culturing a Mycoplasma, a primer capable of amplifying a nucleic acid sequence derived from a Mycoplasma, and a filter for removing impurities.

The methods and other aspects disclosed here will be described in more detail with reference to the following examples; however, these are examples and the present invention is not limited thereto.

### Examples

I. Material

1. Mycoplasma Strain

The following seven kinds of Mycoplasmas were used based on The Japanese Pharmacopoeia 17th Edition (JP17) General Information. Incidentally, all kinds thereof were available from public storage institute and those which had been passaged five times or less were used.

Mycoplasma arginini ATCC23838

Mycoplasma salivarium NBRC14478

Mycoplasma pneumoniae NBRC14401

Mycoplasma fermentans NBRC14854

Mycoplasma orale NBRC14477

Mycoplasma hyorhinis NBRC14858

Acholeplasma laidlawii NBRC14400

2. Liquid Culture Medium for Mycoplasma Detection Test

Heart Infusion Broth, glucose, arginine hydrochloride, and phenol red were mixed so as to be 18.75 g/L, 3.0 g/L, 3.0 g/L, and 0.025 g/L at a final concentration, respectively, and then autoclave sterilization was performed at 121° C. for 15 minutes. A horse serum, a yeast extract, and potassium penicillin G were added thereto so as to be 15%, 10%, and 250,000 units/L at a final concentration, respectively.

3. Agar Medium for Mycoplasma Amplification

Heart Infusion Broth and agar were mixed so as to be 17.5 g/L and 10 g/L at a final concentration, respectively, and then autoclave sterilization was performed at 121° C. for 15 minutes. A horse serum and a yeast extract were added thereto so as to be 20% and 10% at a final concentration, respectively.

4. Specimen

As for the specimen, cryopreserved cells, a sheet preparation culture medium, and a cell washing solution were used. The cryopreserved cells were cells obtained by suspending skeletal myoblasts in a cryopreservation liquid in a concentration of 3.0×10⁷ cells/mL and then freezing the suspended cells. In the following experiment, cryopreserved cells, which were gently shaken up in a water bath set at 37.0° C. and then melted, were provided to the test. The sheet preparation culture medium was obtained by mixing a DMEM/F12 culture medium and human-derived serum in an amount of 20% (v/v). The cell washing solution was obtained by mixing a Hanks' buffered saline solution (HBSS(-)) and human serum albumin in an amount of 0.5% (v/v). Hereinafter, the “cryopreserved cells” are also simply referred to as the “cells”, the “sheet preparation culture medium” is also simply referred to as the “culture medium”, and the “cell washing solution” is also simply referred to as the “washing solution”.

II. Method and result

1. Setting of Impurity Removing Step

100 μL of cells, which had been checked that live Mycoplasmas were not mixed, were added as a negative control to 2 mL of a liquid culture medium for a Mycoplasma detection test, and then subjected to aerobic cultivation for 7 days under the conditions of 36° C. and 5% CO₂. Further, as a positive control, one obtained by adding 100 μL of cells and 10 μL of a Mycoplasma diluent containing 7500 cfu/μL of Acholeplasma laidlawii to 2 mL of a liquid culture medium for a Mycoplasma detection test was subjected to aerobic cultivation.

DNA extraction was performed by using MagNA Pure LC DNA Isolation Kit-Large Volume (Roche) to finally obtain a 200 μL DNA extract. 90 μL of PCR reaction solution (Takara Ex taq) was added to 10 μL of the DNA extract and PCR was performed. As for PCR, PCR was performed for one cycle of 5 minutes at 40° C., one cycle of 10 minutes at 94° C., 20 cycles of 30 seconds at 94° C., 30 seconds at 70° C., and 45 seconds at 72° C. (the part at 70° C. decreases by 1° C. per two cycles, up to 61° C.), 25 cycles of 30 seconds at 94° C., 30 seconds at 60° C., and 45 seconds at 72° C., and one cycle of 4 minutes at 72° C. As for the primer, the following sequences were used.

Primer sequences:

Forward:
(SEQ ID NO: 1)
5′-GGCGAATGGGTGAGTAACACG-3′
Reverse:
(SEQ ID NO: 2)
5′-CGGATAACGCTTGCGACCTATG-3′

The reaction solution after PCR was subjected to electrophoresis with an agarose gel, and then the presence or absence of a DNA band was checked.

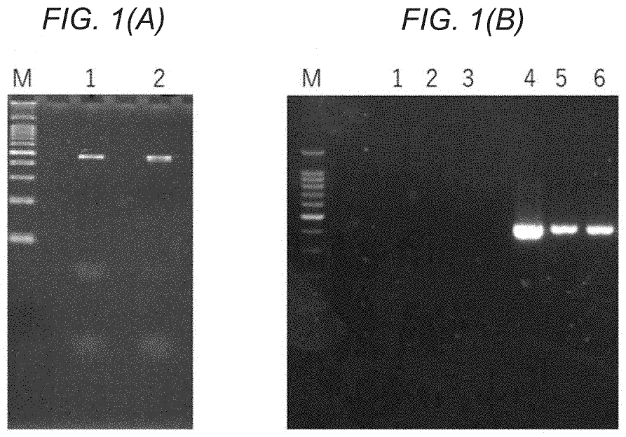

The result is shown in FIG. 1(A). M shows a 100 bp ladder, Lane 1 shows the result when PCR is performed without addition of a Mycoplasma, and Lane 2 shows a positive control (the result when PCR is performed with addition of a Mycoplasma). Even in a case where a Mycoplasma was not added (Lane 1), the band having the same size as that of a case where a Mycoplasma is added (positive control, Lane 2) appeared. Since cells, which had been checked that live Mycoplasmas were not mixed, were used as a specimen, it was considered that nucleic acid fragments of the Mycoplasma were mixed in this test system.

In order to remove the nucleic acid fragments of the Mycoplasma from the test system, trapping of the Mycoplasma by a centrifugal ultrafiltration filter was examined.

100 μL of cells, which had been checked that live Mycoplasmas were not mixed, were added as a negative control to 2 mL of a liquid culture medium for a Mycoplasma detection test, and then subjected to aerobic cultivation for 7 days under the conditions of 36° C. and 5% CO₂. Further, as a positive control, one obtained by adding 100 μL of cells and 10 μL of a Mycoplasma diluent containing 7500 cfu/μL of Acholeplasma laidlawii to 2 mL of a liquid culture medium for a Mycoplasma detection test was subjected to aerobic cultivation.

500 μL of the supernatant subjected to centrifugation for 5 minutes at 4° C. and 400×g was transferred to a centrifugal ultrafiltration filter (Merck Millipore, Am icon Ultra 100 kDa) having a molecular weight cutoff of 100 kDa and then subjected to centrifugation for 10 minutes at 4° C. and 14,000×g. 500 μL of PBS was added thereto and subjected to centrifugation for 10 minutes at 4° C. and 14,000×g, 500 μL of PBS was then further added and subjected to vigorously pipetting, and the solution was recovered. The reaction solution after PCR was subjected to electrophoresis with an agarose gel and then the presence or absence of a DNA band was checked.

The result is shown in FIG. 1(B). M shows a 100 bp ladder, Lanes 1 to 3 show the result when PCR is performed without addition of a Mycoplasma after collection with a filter, and Lanes 4 to 6 show a positive control (the result when PCR is performed with addition of a Mycoplasma after collection with a filter). In the case of the negative control (Lanes 1 to 3), bands did not appear, and only in a case where a Mycoplasma was added (positive control, Lanes 4 to 6), bands appeared. As described above, through the centrifugal ultrafiltration filter, it was shown that DNA fractions of the Mycoplasma can be removed from the test system.

2. Selection of Filter

A centrifugal ultrafiltration filter for trapping a Mycoplasma was examined. As for the centrifugal ultrafiltration filter, three types of a centrifugal ultrafiltration filter having a molecular weight cutoff of 100 kDa (Merck Millipore, Am icon Ultra 100 kDa), a centrifugal ultrafiltration filter having a pore size of 0.1 μm (Merck Millipore, Ultrafree 0.1 μm), and a centrifugal ultrafiltration filter having a pore size of 0.22 μm (Merck Millipore, Ultrafree 0.22 μm) were used, and as for the Mycoplasma, Mycoplasma pneumoniae and Acholeplasma laidlawii were used. As the specimen, a cell, a culture medium, and a washing solution were used. In a case where the specimen is a cell, 2000 μL of a liquid culture medium for a Mycoplasma detection test, 100 μL of a specimen, and 100 μL of 20000 cfu/mL Mycoplasma diluent were added. 500 μL of the supernatant subjected to centrifugation for 5 minutes at 4° C. and 400×g was transferred to a filter and then subjected to centrifugation for 10 minutes at 4° C. and 14,000×g. In a case where the specimen is a culture medium or a washing solution, 2000 μL of a liquid culture medium for a Mycoplasma detection test, 500 μL of a specimen, and 100 μL of 20000 cfu/mL Mycoplasma diluent were added. 500 μL was transferred to a filter and then subjected to centrifugation for 10 minutes at 4° C. and 14,000×g. 500 μL of PBS was added thereto and subjected to centrifugation for 10 minutes at 4° C. and 14,000×g, 500 μL of PBS was then further added and subjected to vigorously pipetting, and the Mycoplasma trapped with the filter was recovered. As a negative control, the same operation was performed in the condition without addition of a Mycoplasma diluent.

DNA was extracted from the filter-recovered liquid by using MagNA Pure LC DNA Isolation Kit-Large Volume (Roche) to finally obtain a 200 μL DNA extract.

10 μL of the DNA extract was added to 90 μL of PCR reaction solution (Takara Ex taq), and PCR was performed for one cycle of 5 minutes at 40° C., one cycle of 10 minutes at 94° C., 20 cycles of 30 seconds at 94° C., 30 seconds at 70° C., and 45 seconds at 72° C. (the part at 70° C. decreases by 1° C. per two cycles, up to 61° C.), 25 cycles of 30 seconds at 94° C., 30 seconds at 60° C., and 45 seconds at 72° C., and one cycle of 4 minutes at 72° C. As for the primer, the following sequences were used.

Primer sequences:

Forward:
(SEQ ID NO: 1)
5′-GGCGAATGGGTGAGTAACACG-3′
Reverse:
(SEQ ID NO: 2)
5′-CGGATAACGCTTGCGACCTATG-3′

The reaction solution after PCR was subjected to electrophoresis with an agarose gel, and then the presence or absence of a DNA band was checked. A case where a DNA band of the Mycoplasma can be confirmed in the filter-recovered liquid was determined that the Mycoplasma can be trapped. The results are shown in Table 1. As for Mycoplasma pneumoniae, the Mycoplasma was able to be trapped in any specimens with a filter having a pore size of 0.22 μm. As for Acholeplasma laidlawii, the Mycoplasma was able to be trapped in the washing solution specimen with a filter having a pore size of 0.22 μm, the Mycoplasma was able to be trapped in the culture medium specimen with a filter having a pore size of 0.1 μm, and the Mycoplasma was able to be trapped in the cellular specimen with a filter having a molecular weight cutoff of 100 kDa. In all the specimens, only in the case of using a filter having a molecular weight cutoff of 100 kDa, two kinds of Mycoplasmas were able to be trapped.

### [Table 1]

TABLE 1
Trapping possibility of mycoplasma in each filter

| | | Nega- | | |
|---|---|---|---|---|
| | | tive | Acholeplassma | Mycoplasma |
| Filter | Specimen | Control | laidlawii | pneumoniae |
| AmiconUltra | Cell | − | + | + |
| 100 kDa | Culture medium | − | + | + |
| | Washing solution | − | + | + |
| Ultrafree | Cell | − | − | + |
| 0.1 μm | Culture medium | − | + | + |
| | Washing solution | − | + | + |
| Ultrafree | Cell | − | + | + |
| 0.22 μm | Culture medium | − | − | + |
| | Washing solution | − | + | + |

+: trapping is possible,
−: trapping is impossible

3. Checking of Proliferation of Mycoplasma

As for seven kinds of Mycoplasmas, it was checked how much the Mycoplasma was amplified when the Mycoplasma was added and cultured so as to have a concentration of 10 cfu/mL or less in the respective specimens of the cells, the culture medium, and the washing solution.

In a case where the specimen is a cryopreserved cell, 2 mL of a liquid culture medium for a Mycoplasma detection test, 100 μL of a specimen, and 10 μL of a Mycoplasma diluent prepared to contain a desired Mycoplasma were added to a well. The Mycoplasma was added such that 0.80 cfu of the Mycoplasma was contained in the case of testing Mycoplasma arginini, 0.54 cfu of the Mycoplasma was contained in the case of testing Mycoplasma salivarium, 0.68 cfu of the Mycoplasma was contained in the case of testing Mycoplasma pneumoniae, 0.60 cfu of the Mycoplasma was contained in the case of testing Mycoplasma fermentans, 0.60 cfu of the Mycoplasma was contained in the case of testing Mycoplasma orale, 0.74 cfu of the Mycoplasma was contained in the case of testing Mycoplasma hyorhinis, and 0.84 cfu the Mycoplasma was contained in the case of testing Acholeplasma laidlawii, per well. That is, the Mycoplasma was added such that the Mycoplasma would be 0.80 cfu/100 μL (8.0 cfu/mL) in the case of testing Mycoplasma arginini, 0.54 cfu/100 μL (5.4 cfu/mL) in the case of testing Mycoplasma salivarium, 0.68 cfu/100 μL (6.8 cfu/mL) in the case of testing Mycoplasma pneumoniae, 0.60 cfu/100 μL (6.0 cfu/mL) in the case of testing Mycoplasma fermentans, 0.60 cfu/100 μL (6.0 cfu/mL) in the case of testing Mycoplasma orale, 0.74 cfu/100 μL (7.4 cfu/mL) in the case of testing Mycoplasma hyorhinis, and 0.84 cfu/100 μL (8.4 cfu/mL) in the case of testing Acholeplasma laidlawii, per 100 μL of the specimen.

In a case where the specimen is a sheet preparation culture medium or a cell washing solution, 2 mL of a liquid culture medium for a Mycoplasma detection test, 500 μL of a specimen, and 50 μL of a Mycoplasma diluent prepared to contain a desired Mycoplasma were added to a well. The Mycoplasma was added such that 4.0 cfu of the Mycoplasma was contained in the case of testing Mycoplasma arginini, 2.7 cfu of the Mycoplasma was contained in the case of testing Mycoplasma salivarium, 3.4 cfu of the Mycoplasma was contained in the case of testing Mycoplasma pneumoniae, 3.0 cfu of the Mycoplasma was contained in the case of testing Mycoplasma fermentans, 3.0 cfu of the Mycoplasma was contained in the case of testing Mycoplasma orale, 3.7 cfu of the Mycoplasma was contained in the case of testing Mycoplasma hyorhinis, and 4.2 cfu the Mycoplasma was contained in the case of testing Acholeplasma laidlawii, per well. That is, the Mycoplasma was added such that the Mycoplasma would be 4.0 cfu/500 μL (8.0 cfu/mL) in the case of testing Mycoplasma arginini, 2.7 cfu/500 μL (5.4 cfu/mL) in the case of testing Mycoplasma salivarium, 3.4 cfu/500 μL (6.8 cfu/mL) in the case of testing Mycoplasma pneumoniae, 3.0 cfu/500 μL (6.0 cfu/mL) in the case of testing Mycoplasma fermentans, 3.0 cfu/500 μL (6.0 cfu/mL) in the case of testing Mycoplasma orale, 3.7 cfu/500 μL (7.4 cfu/mL) in the case of testing Mycoplasma hyorhinis, and 4.2 cfu/500 μL (8.4 cfu/mL) in the case of testing Acholeplasma laidlawii, per 500 μL of the specimen.

Six kinds of Mycoplasmas excluding Mycoplasma salivarium were subjected to aerobic cultivation under the conditions of 36° C. and 5% CO₂, and Mycoplasma salivarium was subjected to anaerobic cultivation under the condition of 36° C. After 3 days from the cultivation, the culture solution was extracted from each well after 7 days, and the number of bacteria was measured.

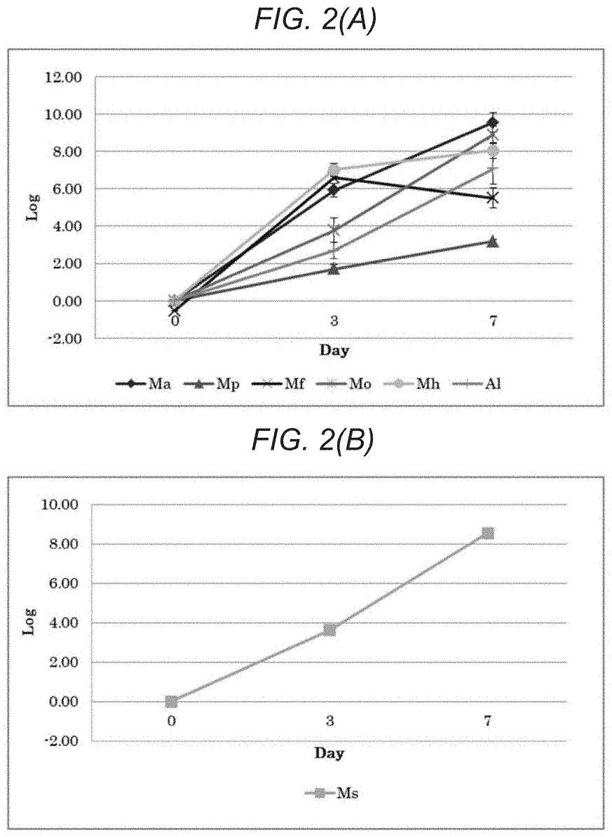

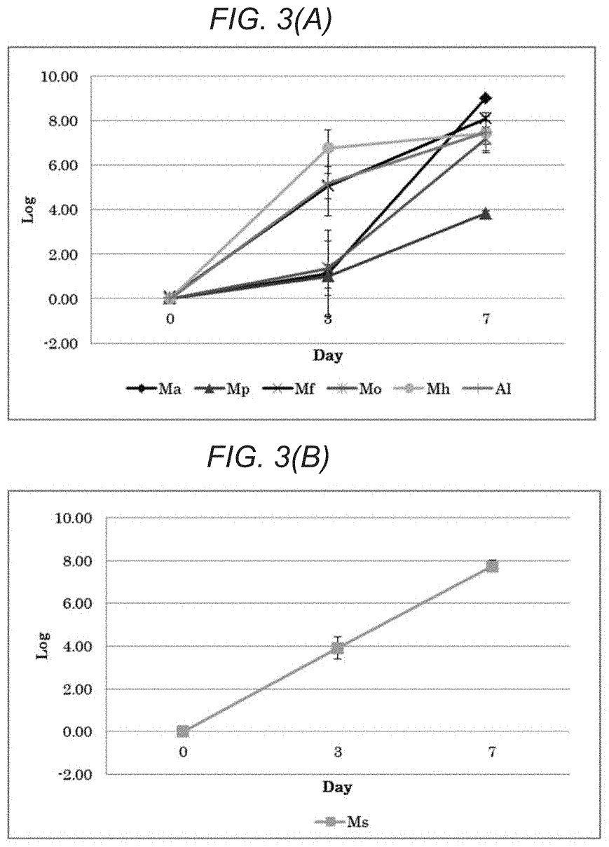

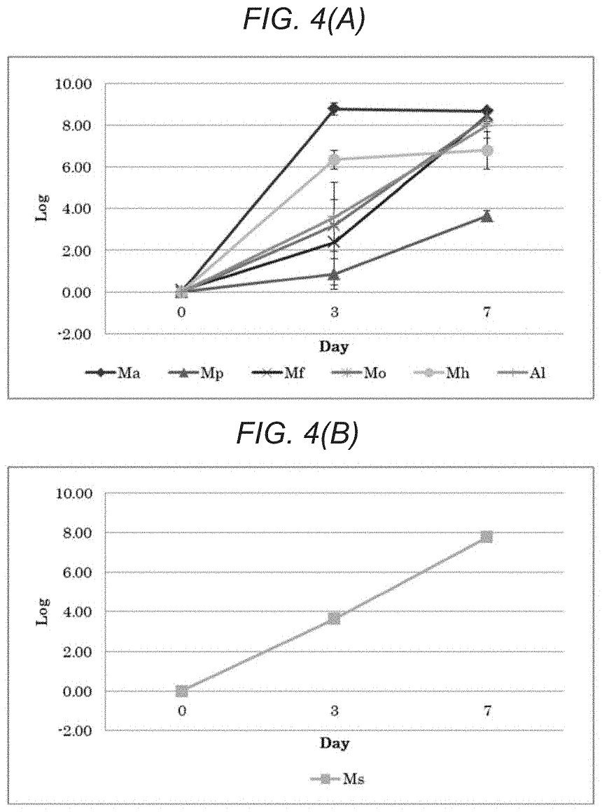

The results are shown in FIGS. 2 to 4. The amount of the Mycoplasma after the cultivation for 7 days was increased to 1500 to 10⁹ cfu/mL although varying depending on the type of bacteria, and in this precultivation step, it was confirmed that a small amount of the Mycoplasma is increased to a sufficient amount.

4. Calculation of Detection Limit

In respective specimens of the cells, the culture medium, and the washing solution, detection limits of seven kinds of Mycoplasmas were calculated.

In a case where the specimen is a cryopreserved cell, 852 μL of a liquid culture medium for a Mycoplasma detection test and 48 μL of a specimen were added to a microtube, and 100 μL of a Mycoplasma diluent prepared to contain a desired Mycoplasma was added thereto. The Mycoplasma was added so as to be 1000 cfu, 500 cfu, 100 cfu, and 50 cfu per microtube. That is, with respect to the total volume in the microtube, the Mycoplasma was added so as to have concentrations of 1000 cfu/mL, 500 cfu/mL, 100 cfu/m L, and 50 cfu/mL.

In a case where the specimen is a sheet preparation culture medium or a cell washing solution, 525 μL of a liquid culture medium for a Mycoplasma detection test, 150 μL of a specimen, and 75 μL of a Mycoplasma diluent prepared to contain a desired Mycoplasma were added to a microtube. The Mycoplasma was added so as to be 750 cfu, 375 cfu, 75 cfu, and 37.5 cfu per microtube. That is, with respect to the total volume in the microtube, the Mycoplasma was added so as to have concentrations of 1000 cfu/m L, 500 cfu/m L, 100 cfu/m L, and 50 cfu/mL.

In a case where the specimen is a cryopreserved cell, 500 μL of the supernatant subjected to centrifugation for 5 minutes at 4° C. and 400×g was transferred to a centrifugal ultrafiltration filter (Merck Millipore) having a molecular weight cutoff of 100 kDa and then subjected to centrifugation for 10 minutes at 4° C. and 14,000×g.

In a case where the specimen is a culture medium or a washing solution, 500 μL was transferred to a centrifugal ultrafiltration filter (Merck Millipore) having a molecular weight cutoff of 100 kDa and then subjected to centrifugation for 10 minutes at 4° C. and 14,000×g. 500 μL of PBS was added thereto and subjected to centrifugation for 10 minutes at 4° C. and 14,000×g, 500 μL of PBS was then further added and subjected to vigorously pipetting, and the Mycoplasma trapped with the filter was recovered.

DNA was extracted from the total amount of the recovered liquid after removing impurities by using MagNA Pure LC DNA Isolation Kit-Large Volume (Roche) to finally obtain a 200 μL DNA extract.

10 μL of the DNA extract was added to 90 μL of PCR reaction solution (Takara Ex taq), and PCR was performed for one cycle of 5 minutes at 40° C., one cycle of 10 minutes at 94° C., 20 cycles of 30 seconds at 94° C., 30 seconds at 70° C., and 45 seconds at 72° C. (the part at 70° C. decreases by 1° C. per two cycles, up to 61° C.), 25 cycles of 30 seconds at 94° C., 30 seconds at 60° C., and 45 seconds at 72° C., and one cycle of 4 minutes at 72° C. As for the primer, the following sequences were used.

Primer sequences:

Forward:
(SEQ ID NO: 1)
5′-GGCGAATGGGTGAGTAACACG-3′
Reverse:
(SEQ ID NO: 2)
5′-CGGATAACGCTTGCGACCTATG-3′

The reaction solution after PCR was subjected to electrophoresis with an agarose gel, and then the presence or absence of a DNA band was checked.

Calculation was performed 24 times for respective Mycoplasma concentrations of 1000 cfu/m L, 500 cfu/m L, 100 cfu/m L, and 50 cfu/m L, and from the ratio of the Mycoplasma concentration and the number of positive cells (the number of cells in which a DNA band specific to the Mycoplasma was confirmed), probit analysis was performed using NATIONAL INSTITUTE OF INFECTIOUS DISEASE statistical analysis support software Bioassay Assist, and 95% detection sensitivity was calculated. The results are shown in Table 2. The detection limit of each Mycoplasma was 92 to 5647 cfu/m L.

### [Table 2]

TABLE 2
Detection limit

| Specimen | M. arginini | M. salivarium | M. pneumoniae | M. fermentans |
|---|---|---|---|---|
| Cell | 132 cfu/ml | 202 cfu/mL | 1043 cfu/mL | 176 cfu/mL |
| Culture | 335 cfu/mL | 474 cfu/mL | 542 cfu/mL | 124 cfu/mL |
| medium | | | | |
| Washing | 473 cfu/mL | 104 cfu/mL | 502 cfu/mL | 92 cfu/mL |
| solution | | | | |

| Specimen | M. orale | M. hyorhinis | A. laidlawii |
|---|---|---|---|
| Cell | 1365 cfu/mL | 1068 cfu/mL | 3044 cfu/mL |
| Culture | 751 cfu/mL | 1091 cfu/mL | 2579 cfu/mL |
| medium | | | |
| Washing | 633 cfu/mL | 544 cfu/mL | 5647 cfu/mL |
| solution | | | |

5. Detectability of Mycoplasma

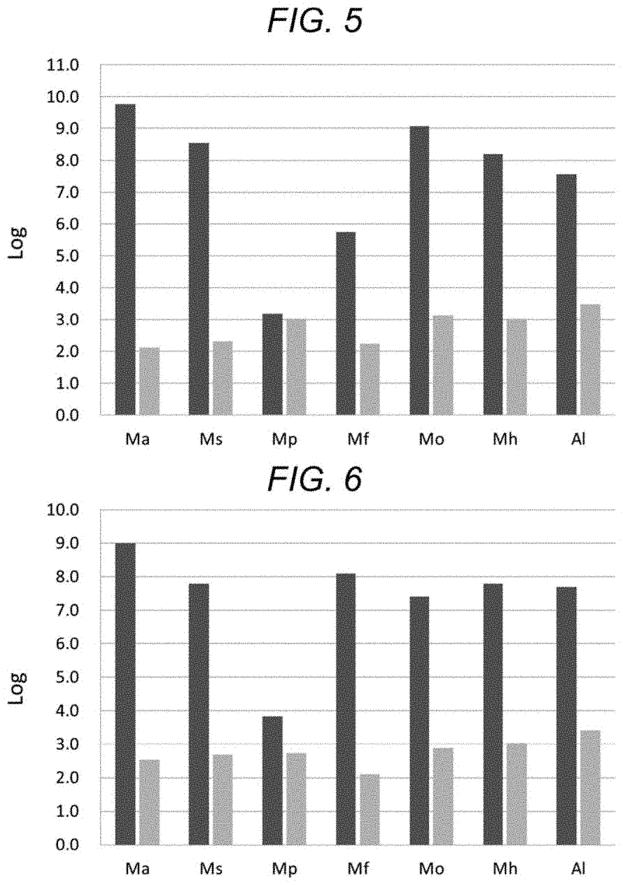

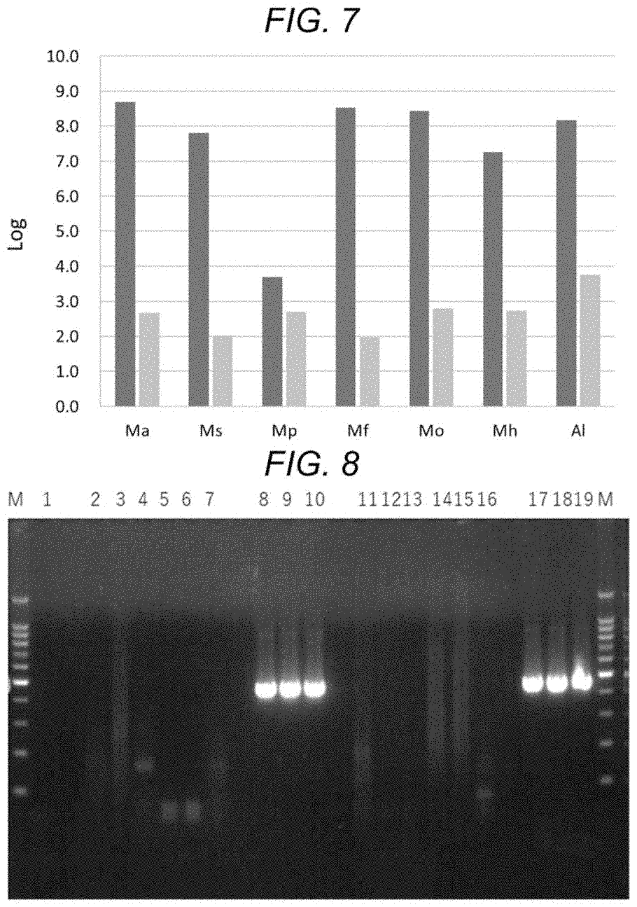

In the respective specimens of the cells, the culture medium, and the washing solution, the amount of the Mycoplasma amplified in the precultivation was compared with the detection sensitivity in this method, and it was verified whether the Mycoplasma contamination can be detected with a high sensitivity as the final test method. The amount of the Mycoplasma amplified in the precultivation and the detection sensitivity in this method are shown in Table 3 and FIGS. 5 to 7. In all the seven kinds of Mycoplasmas, the amount of the Mycoplasma amplified in the precultivation which was calculated exceeded the detection sensitivity calculate. Therefore, in all the seven kinds of Mycoplasmas, it was verified that the Mycoplasma can be detected with a sensitivity of 10 cfu/mL or less from 100 μL of the specimen containing 1 cfu or less of a Mycoplasma in the case of the cellular specimen and from 500 μL of the specimen containing 5 cfu or less of a Mycoplasma in the case of the culture medium and the washing solution specimens.

### [Table 3]

TABLE 3
Detectability of mycoplasma

| | | | | | Detecting |
|---|---|---|---|---|---|
| | | Mycoplasma | Culture | | possibility |
| | | contamination | solution | | (possible: |
| | | per 1 mL of | after | Detection | A > B) |
| | | specimen | precultivation | sensitivity | (impossible: |
| Specimen | Mycoplasma | (cfu/mL) | (A) (cuf/mL) | (B) (cfu/mL) | A < B) |
| Cell | M. arginini | 8.0 | 5.79 × 10⁹ | 132 | Possible |
| | M. salivarium | 5.4 | 3.53 × 10⁸ | 202 | Possible |
| | M. pneumoniae | 6.8 | 1.54 × 10³ | 1043 | Possible |
| | M. fermentans | 6.0 | 5.43 × 10⁵ | 176 | Possible |
| | M. orale | 6.0 | 1.17 × 10⁹ | 1365 | Possible |
| | M. hyorhinis | 7.4 | 1.58 × 10⁸ | 1068 | Possible |
| | A. laidlawii | 8.4 | 3.62 × 10⁷ | 3044 | Possible |
| Culture | M. arginini | 8.0 | 1.00 × 10⁹ | 335 | Possible |
| medium | M. salivarium | 5.4 | 6.18 × 10⁷ | 474 | Possible |
| | M. pneumoniae | 6.8 | 6.77 × 10³ | 542 | Possible |
| | M. fermentans | 6.0 | 1.24 × 10⁸ | 124 | Possible |
| | M. orale | 6.0 | 2.56 × 10⁷ | 751 | Possible |
| | M. hyorhinis | 7.4 | 6.20 × 10⁷ | 1091 | Possible |
| | A. laidlawii | 8.4 | 4.98 × 10⁷ | 2579 | Possible |
| Washing | M. arginini | 8.0 | 4.82 × 10⁸ | 473 | Possible |
| solution | M. salivarium | 5.4 | 6.22 × 10⁷ | 104 | Possible |
| | M. pneumoniae | 6.8 | 4.85 × 10³ | 502 | Possible |
| | M. fermentans | 6.0 | 3.38 × 10⁸ | 92 | Possible |
| | M. orale | 6.0 | 2.71 × 10⁸ | 633 | Possible |
| | M. hyorhinis | 7.4 | 1.84 × 10⁷ | 544 | Possible |
| | A. laidlawii | 8.4 | 1.44 × 10⁸ | 5647 | Possible |

6. Mycoplasma Detection Test

6.1. Precultivation

500 μL of a washing solution was added as a specimen to 2 mL of a liquid culture medium for a Mycoplasma detection test and then subjected to aerobic cultivation for 7 days under the conditions of 36° C. and 5% CO₂. Similarly, 500 μL of a washing solution was added as a specimen to 2 mL of a liquid culture medium for a Mycoplasma detection test and then subjected to anaerobic cultivation for 7 days under the condition of 36° C. As for a negative control, PBS was added instead of a specimen and the same operation was performed. Further, as a positive control, one obtained by adding 50 μL of a Mycoplasma diluent containing 1500 cfu/μL of Mycoplasma orale was subjected to aerobic cultivation, and one obtained by adding 50 μL of a Mycoplasma diluent containing 1500 cfu/μL of Mycoplasma salivarium was subjected to anaerobic cultivation.

6.2. Impurity Removal

500 μL of a culture solution was transferred to a centrifugal ultrafiltration filter (Merck Millipore) having a molecular weight cutoff of 100 kDa and then subjected to centrifugation for 10 minutes at 4° C. and 14,000×g. 500 μL of PBS was added thereto and subjected to centrifugation for 10 minutes at 4° C. and 14,000×g, 500 μL of PBS was then further added and subjected to vigorously pipetting, and the Mycoplasma trapped with the filter was recovered.

6.3. DNA Extraction

DNA was extracted from the total amount of the recovered liquid after removing impurities by using MagNA Pure LC DNA Isolation Kit-Large Volume (Roche) to finally obtain a 200 μL DNA extract.

6.4. PCR and Electrophoresis

10 μL of the DNA extract was added to 90 μL of PCR reaction solution (Takara Ex taq), and PCR was performed for one cycle of 5 minutes at 40° C., one cycle of 10 minutes at 94° C., 20 cycles of 30 seconds at 94° C., 30 seconds at 70° C., and 45 seconds at 72° C. (the part at 70° C. decreases by 1° C. per two cycles, up to 61° C.), 25 cycles of 30 seconds at 94° C., 30 seconds at 60° C., and 45 seconds at 72° C., and one cycle of 4 minutes at 72° C. As for the primer, the following sequences were used.

Primer sequences:

Forward:
(SEQ ID NO: 1)
5′-GGCGAATGGGTGAGTAACACG-3′
Reverse:
(SEQ ID NO: 2)
5′-CGGATAACGCTTGCGACCTATG-3′

The reaction solution after PCR was subjected to electrophoresis with an agarose gel, and then the presence or absence of a DNA band was checked.

6.5. Result

The result is shown in FIG. 8. M shows a 100 bp ladder, Lane 1 shows a negative control (the result when PCR is performed with respect to water), Lanes 2 to 4 show a negative control (the result when PCR is performed by aerobic cultivation using PBS instead of a specimen), Lanes 5 to 7 show the result when PCR is performed by aerobic cultivation of a specimen, Lanes 8 to 10 show a positive control (the result when PCR is performed by aerobic cultivation with addition of a Mycoplasma diluent (Mycoplasma orale) to a specimen), Lanes 11 to 13 show a negative control (the result when PCR is performed by anaerobic cultivation using PBS instead of a specimen), Lanes 14 to 16 show the result when PCR is performed by anaerobic cultivation of a specimen, and Lanes 17 to 19 show a positive control (the result when PCR is performed by anaerobic cultivation with addition of a Mycoplasma diluent (Mycoplasma salivarium) to a specimen). Bands appeared in Lanes 8 to 10 and 17 to 19 as positive controls, and bands did not appear in Lanes 2 to 4 and 11 to 13 as negative controls. Since bands did not appear even in Lanes 5 to 7 and 14 to 16 in which the specimen flowed, it was checked that the Mycoplasma did not exist in the specimen.

The detailed description above describes versions of methods for testing for the presence or absence of a Mycoplasma in a specimen, methods of managing quality of a biotechnology-related product, and kits for use in these methods, representing examples of the inventive methods and kits. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A method for preparing a sample for testing for the presence or absence of a Mycoplasma in a specimen, comprising
culturing a sample from the specimen in a culture medium containing arginine and glucose for Mycoplasma proliferation,
wherein the culturing comprises aerobic culturing of a first sample from the specimen and anaerobic culturing of a second sample from the specimen which is different from the first sample,
providing the first and second samples for detecting seven species of Mycoplasmas to be applied for nucleic acid amplification.

2. The method of claim 1, wherein the culture medium further comprise Heart infusion broth (HIB) and a yeast extract.

3. The method of claim 1, wherein a time period of the culturing is 3 to 14 days.

4. The method of claim 1, wherein the sensitivity of the Mycoplasma detection is 10cfu/mL.

5. The method of claim 1, wherein the specimen is a biotechnology-related product, regenerative medicine-related product or regenerative medicine product.

6. A method for preparing a sample for testing for the presence or absence of a Mycoplasma in a specimen, comprising
culturing a sample of 950 μL or less from the specimen in the culture medium containing arginine and glucose for Mycoplasma proliferation,
providing the first and second samples for detecting seven species of Mycoplasmas applied for nucleic acid amplification.

7. The method of claim 6, wherein the culture medium further comprise Heart infusion broth (HIB) and a yeast extract.

8. The method of claim 6, wherein a time period of the culturing is 3 to 14 days.

9. The method of claim 8, wherein a time period of the culturing is 9 days

10. The method of claim 6, wherein the sample is 100 μL to 500 μL.

11. The method of claim 6, wherein the sensitivity of the Mycoplasma detection is 10cfu/mL.

12. The method of claim 6, wherein the specimen is a biotechnology-related product, regenerative medicine-related product or regenerative medicine product.

13. A method for testing for a presence or absence of a Mycoplasma in a specimen, the method comprising:
culturing a specimen to obtain a cultured specimen, the specimen being a biotechnology-related product or a part of a biotechnology-related product;
extracting a nucleic acid from the cultured specimen; and
performing amplification of the nucleic acid using a primer that is specifically selected to amplify a nucleic acid sequence derived from a Mycoplasma.

14. The method according to claim 13, wherein amplification of the nucleic acid is performed by a polymerase chain reaction (PCR) method.

15. The method according to claim 13, wherein the culturing of the specimen comprises culturing two or more specimens each in an aerobic or anaerobic condition.

16. The method according to claim 13, wherein the culturing of the specimen comprises performing cultivation of the specimen for 3 to 14 days.

17. The method according to claim 13, further comprising removing impurities before the extracting of the nucleic acid with a filter.

18. The method according to claim 13, wherein the regenerative medicine-related product includes at least one of (i) a cell subjected to processing, (ii) a cell to be provided to processing, (iii) a reagent to be used in processing or testing, (iv) a culture medium for culturing a cell, (v) a cell washing solution for washing a cell, and/or (vi) a preservation liquid for preserving a regenerative medicine product.

19. A method of managing quality of a biotechnology-related product, the method comprising:
testing for presence or absence of a Mycoplasma in a specimen of claim 13.

20. A kit for use in the method according to claim 13, the kit comprising: a culture medium for culturing a Mycoplasma; and a primer specifically selected to amplify a nucleic acid sequence derived from Mycoplasma.

21. The kit according to claim 20, further comprising a filter for removing impurities.


SEQUENCE LISTING (2 sequences)

SEQ ID NO: 1 - DNA, 21 bp, Artificial Sequence
  Forward primer for Mycoplasma
ggcgaatgggtgagtaacacg

SEQ ID NO: 2 - DNA, 22 bp, Artificial Sequence
  Reverse primer for Mycoplasma
cggataacgcttgcgacctatg